(19) United States
(12) Patent Application Publication
(10) Pub. No.: US 2025/0073275 A1
(43) Pub. Date: Mar. 6, 2025

(54) THERAPEUTIC AGENT-LOADED TISSUES FOR CORNEAL TRANSPLANT

(71) Applicant: University of South Florida, Tampa, FL (US)

(72) Inventors: Christopher L. Passaglia, Lutz, FL (US); Edgar España, Tampa, FL (US); Emily Lynn Checchio, Destin, FL (US)

(21) Appl. No.: 18/822,769

(22) Filed: Sep. 3, 2024

Related U.S. Application Data
(60) Provisional application No. 63/580,267, filed on Sep. 1, 2023.

(51) Int. Cl. A61K 35/30; A61K 9/50; A61K 45/06; C12N 5/079

(52) U.S. Cl. CPC A61K 35/30; A61K 9/5063; A61K 45/06; C12N 5/0621

(57) ABSTRACT

Disclosed herein are methods of loading therapeutic agents into corneal tissues, corneal tissues including therapeutic agents, and surgical methods including keratoplasties in which corneal tissues having one or more therapeutic agents loaded therein are transplanted into a patient.

### CROSS-REFERENCE TO RELATED APPLICATION

This application claims the benefit of U.S. Provisional Application 63/580,267, filed Sep. 1, 2023, the contents of which are hereby incorporated in its entirety.

### BACKGROUND

A cornea is the eye's outermost layer, and it is an essential component of ocular anatomy. Under normal conditions, it consists of a clear, dome-shaped surface that covers the anterior portion of the eye and serves as a protective barrier. The cornea is comprised of five tissue layers, ordered from the outermost to the innermost layer: the Epithelium, Bowman's layer, Stroma, Descemet's membrane, and the Endothelium. These tissue layers collectively maintain the cornea's transparency and refractive properties. Medical intervention in the form of corneal transplantation may be necessary to restore vision and alleviate visual impairments caused by corneal abnormalities, diseases, or injuries that cannot be effectively treated through other means. In the case of a full thickness transplant, all layers of the affected cornea are removed. [FIG. 2]. In contrast, a partial thickness transplant entails extracting and replacing either the anterior or posterior layers of the cornea while leaving remaining layers intact. Partial-thickness techniques include: Descemetorhexis without endothelial keratoplasty (DWEK), Descemet stripping only (DSO), Deep lamellar endothelial keratoplasty (DLEK), Descemet stripping endothelial keratoplasty (DSEK), Descemet stripping automated endothelial keratoplasty (DSAEK), Descemet membrane endothelial keratoplasty (DMEK), Pre-Descemet endothelial keratoplasty (PDEK), and Descemet membrane endothelial transfer (DMET).

There are currently two clinical approaches used to transplant corneas, known as partial- and full-thickness keratoplasties. A shared feature of these two keratoplasty methods is an absence of preventative treatment measures pre-transplant. This means that post-transplant issues are addressed as they arise rather than before they occur. Several issues may be anticipated, such as inflammation, infection, and donor tissue rejection, as patients are significantly more susceptible to afflictions during the post-transplant recovery period. Although the standard clinical strategies to treat post-transplant problems can be effective, diseases, inflammation, and infections can contribute to eye stress and strain that can potentially lead to eye damage if left untreated or unmanaged. Some common causes are bacterial, viral, or fungal eye infections, dry eye syndrome, allergic conjunctivitis, keratitis (Upadhyay et al., 2015), glaucoma, and uveitis.

Prior to conducting corneal transplants, it is imperative to assess the quality of the donor cornea tissue and evaluate the viability of the endothelium to determine the feasibility of the transplantation procedure. In general, the criteria for qualifying donor corneas are relatively minimal, indicating that compatibility between a donor and recipient is not restricted by factors such as race, ethnicity, gender, or age. However, factors such as communicable diseases and trauma play a significant role in determining the suitability of donor corneas. Corneas from donors with diseases or injuries can lead to damage in the endothelium, which in turn heightens the risk of corneal transplant rejection. While corneal tissue can be obtained from living donors, it is important to note that the majority of corneal transplants utilize tissue from deceased donors, which can still remain viable for transplantation purposes.

There remains a need for improved systems and methods for corneal transplantation. There remains a need for improved donor corneal tissues. There remains a need for treating and preventing post-transplant complications.

### BRIEF DESCRIPTION OF THE FIGURES

FIG. 1 depicts the five layers of corneal tissue. From the outermost to the innermost layer, they are the epithelium, Bowman's layer, stroma, Descemet's membrane, and the endothelium.

FIG. 2 depicts partial- and full-thickness corneal transplants. A partial cornea transplant, known as lamellar keratoplasty, involves selectively replacing the anterior or posterior layers of the cornea while leaving the remaining layers intact (left). A full-thickness cornea transplant, also known as penetrating keratoplasty, entails the surgical replacement of all layers of the diseased or damaged cornea with a healthy donor cornea (right). The schematic depicts the process of suturing donor cornea tissue into position following the removal of the abnormality or disease from the patient's cornea.

FIG. 3 depicts a schematic representation of an embodiment of the disclosure. 1) Obtain the donor cornea and perform the required incisions for the intended transplant, 2) Apply the medication onto the microneedle array pen, 3) Utilize the microneedle array pen to inject the medication into the donor cornea, and 4) Insert the therapeutic agent-loaded donor cornea into the recipient.

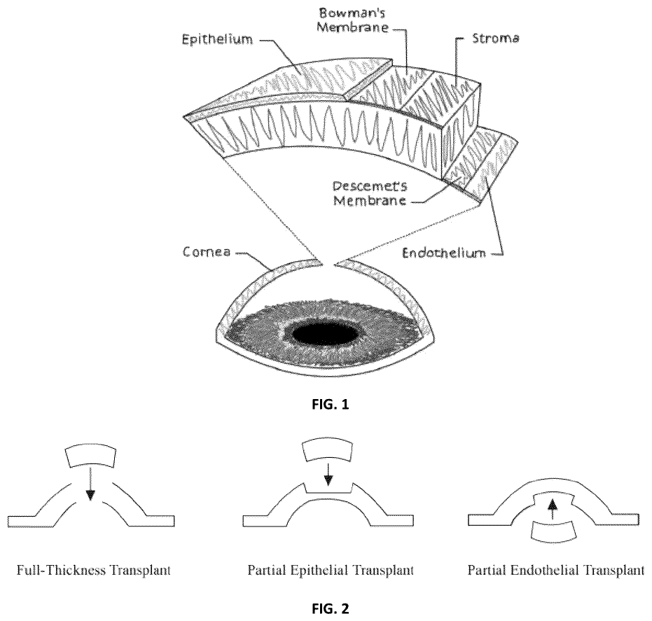

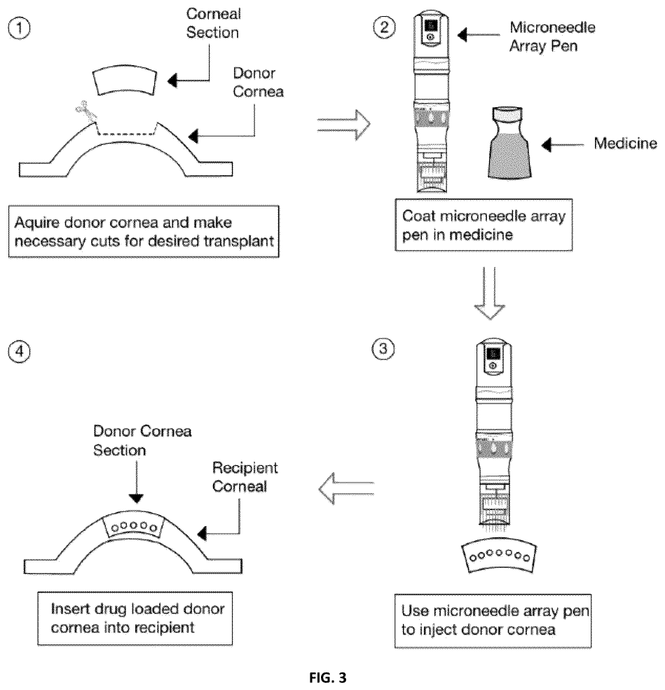

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Disclosed herein are methods of loading one or more therapeutic agents onto corneal tissue. As used herein, loading refers to any process by which a therapeutic agent (defined as a small molecule or biomacromolecule) is associated with corneal cells such that at least a portion of the therapeutic agent is released from the corneal tissue following transplantation. Unless specified to the contrary, use of the term “therapeutic agent” refers both to single therapeutic agents and combinations of two or more therapeutic agents.

In certain implementations, corneal tissue may be obtained from the patient to receive the transplant, a human cadaver, or reconstructed using tissue engineering techniques. Autologous and donor tissues can be cultivated ex vivo prior to therapeutic agent-loading and transplant. The corneal tissue may be obtained using stem cells, for example embryonic stem cells, pluripotent stem cells, mesenchymal stem cells (for example mesenchymal fibroblast stem cells), cells derived from stem cells extracted from the patient (for example limbal epithelial stem cells and/or stromal stem cells), or a combination thereof. Corneal tissue engineering is described in greater detail in Tissue Eng. Part B Rev. (2015) 21(3):278-287, the contents of which are hereby incorporated by reference.

In some implementations, the corneal tissue is a complete corneal tissue which possesses each of the five layers found in normal corneal tissue. In some implementations the corneal tissue lacks one or more of the five layers. Such tissues may be obtained using tissue engineering or surgical dissection. In some implementations, the corneal tissue lacks an endothelial layer. In some implementations the corneal tissue lacks an epithelial layer. In some implementations the corneal tissue lacks both the endothelial and epithelial layers. Such tissues may be used as lamellar or partial thickness replacement for a patient's existing cornea.

In some implementations, the therapeutic agent may be loaded on each layer of corneal tissue. In other implementations, the therapeutic agent is selectively loaded onto one or more layers of corneal tissue. As used herein, selectively loaded refers to a process resulting in 80% of therapeutic agent residing in the identified tissue layers. In some implementations, the therapeutic agent is selectively loaded on the epithelium. In some implementations, the therapeutic agent is selectively loaded on the epithelium and Bowman's layer. In some implementations, the therapeutic agent is selectively loaded on the epithelium, Bowman's layer stroma, and stroma. In some implementations, the therapeutic agent is selectively loaded on the epithelium, Bowman's layer stroma, stroma, and Descemet's membrane. In some implementations, the therapeutic agent is selectively loaded on the endothelium. In some implementations, the therapeutic agent is selectively loaded on the endothelium and Descemet's membrane. In some implementations, the therapeutic agent is selectively loaded on the endothelium, Descemet's membrane, and stroma. In some implementations, the therapeutic agent is selectively loaded on the endothelium, Descemet's membrane, stroma, and Bowman's layer.

The therapeutic agent may be loaded onto corneal tissue by several techniques. In some implementations, the therapeutic agent may be loaded using a microneedle array. The length of the microneedles may be selected to provide delivery to one or more individual layers. In some implementations, the microneedles have a length of 1000 μm or less, 900 μm or less, 800 μm or less, 700 μm or less, 600 μm or less, 500 μm or less, 400 μm or less, 300 μm or less, 200 μm or less, 100 μm or less, or 50 μm or less. In some implementations, the microneedles can have a length from 50-600 μm, from 50-500 μm, from 50-400 μm, from 50-300 μm, from 50-200 μm, 50-100 μm, from 100-500 μm, from 250-500 μm, from 100-250 μm, from 200-400 μm, from 100-300 μm, from 300-600 μm, or from 400-600 μm. In some implementations the microneedles have a length from 150-500 μm. The microneedles may be dipped other otherwise coated with a solution containing one or more therapeutic agents, corneal tissue. The microneedles may be hollow microneedles, and a solution containing therapeutic agent may be loaded into the hollow space. In certain implementations, a microneedle array pen may be used to load a therapeutic agent onto the corneal tissue. In other implementations, a microneedle array may be used to place punctures in corneal tissue, and the punctured tissue is then contacted with a solution containing the therapeutic agent.

In certain implementations, the therapeutic agent may be loaded on the corneal tissue using electroporation (which is alternatively designated electropermeabilization or electrotransfer). In electroporation, corneal tissue is disposed in a solution containing a therapeutic agent and an electrical pulse (usually a direct current) is applied to the corneal tissue for a brief duration of time (milliseconds to seconds), which generates pores in the corneal tissue. The therapeutic agent then enters into the tissue via the pores. Upon cessation of the current the pores close with the therapeutic agent sequestered in the corneal tissue.

In certain implementations, the therapeutic agent may be loaded on the corneal tissue by surgical incision. The technique has similarities to LASIK surgery; in that, a small incision is made to create a corneal flap, the flap is gently lifted, the therapeutic agent is applied to the exposed area, the flap is carefully repositioned, and sutures are applied if needed for proper closure. The therapeutic agent may be topically applied to the tissue via dropper or syringe, or the therapeutic agent may be coated onto the tissue using a spatula or similar implement. In some implementations, the therapeutic agent may be injected into the exposed tissue using a syringe or microneedle array.

In some implementations the therapeutic agent may be loaded into the corneal tissue by solution equilibration methods. The corneal tissue is disposed in a solution including the therapeutic agent, which then passively diffuses into the corneal tissue. In some implementations, the therapeutic agent may be loaded on the corneal tissue by adding the therapeutic agent to the storage media (M-K K media, cornisol media, etc.). In some implementations therapeutic agent is not a growth factor, antibiotic, or antifungal therapeutic agent, for example the therapeutic agent is not gentamicin or amphotericin-B.

In some implementations, the corneal tissue is removed from the storage media and disposed in a separate solution containing the therapeutic agent. The storage media may include the therapeutic agent, or different therapeutic agent, than the therapeutic agent in the separate solution.

In some implementations, corneal endothelial cells (e.g., human corneal endothelial cells) can be cultured in a medium including one or more therapeutic agents. Culture techniques are known in the art, for example described in Stem Cell Res Ther. (2021) 12(1):554, the contents of which are hereby incorporated by reference. In certain implementations, the corneal endothelial cells are cultured in the presence of a therapeutic agent other than a growth factor, antibiotic, or antifungal therapeutic agent, for example a therapeutic agent other than gentamicin or amphotericin-B.

A variety of different therapeutic agents can be loaded into the corneal tissue. In some implementations, the therapeutic agent is an antibacterial, antifungal, antiviral, cycloplegic, antiglaucoma therapy, corticosteroid, immunosuppressant, dry-eye therapy, anesthetic, anti-inflammatory, anti-allergy, β-adrenoceptor antagonist, miotic, carbonic anhydrase inhibitor, topoisomerase inhibitor, prostaglandin, aldose reductase inhibitor, growth factor, collagenase inhibitor, or combination thereof.

In certain implementations, the therapeutic agent is not gentamicin or amphotericin B.

In some implementations the therapeutic agent is betamethasone, clobetasone, dexamethasone, fluorometholone, hydrocortisone, prednisolone, or combination thereof.

In some implementations the therapeutic agent is antazoline, bromfenac, diclofenac, indomethacin, lodoxamide, saprofen, sodium cromoglycate, or combination thereof.

In some implementations the therapeutic agent is amethocaine, lignocaine, oxbuprocaine, proxymetacaine, or combination thereof.

In some implementations the therapeutic agent is cyclosporin, tacrolimus, or sirolimus.

In some implementations the therapeutic agent is pilocarpine, carbachol, physostigmine, or combination thereof.

In some implementations the therapeutic agent is carteolol, cetamolol, betaxolol, levobunolol, metipranolol, timolol, or combination thereof.

In some implementations the therapeutic agent is adrenaline, dipivefrine, or combination thereof.

In some implementations the therapeutic agent is acetazolamide, dorzolamide, or combination thereof.

In some implementations the therapeutic agent is topotecan, irinotecan, camptothecin, lamellarin D, etoposide, teniposide, doxorubicin, mitoxantrone, or amsacrine.

In some implementations the therapeutic agent is chloramphenicol, chlortetracycline, ciprofloxacin, framycetin, fusidic acid, gentamicin, neomycin, norfloxacin, ofloxacin, polymyxin, propamidine, tetracycline, tobramycin, quinolines, or combination thereof.

In some implementations the therapeutic agent is acyclovir, cidofovir, brincidofovir, ganciclovir, valacyclovir, trifluridine, trifluorothymidine, idoxuridine, foscarnet, interferons, or combination thereof.

In some implementations the therapeutic agent is pegagtanib sodium, ranibizumab, aflibercept or bevacizumab.

In some implementations the therapeutic agent is lidocaine, amethocaine, lignocaine, oxbuprocaine, proxymetacaine, or combination thereof.

In some implementations the therapeutic agent is moxifloxacin, gatifloxacin, tobramycin, amikacin, or combination thereof.

In some implementations the therapeutic agent is homotropine or atropine, or combination thereof.

In some implementations the therapeutic agent is natamycin, voriconazole, itraconazole, ketoconazole, or combination thereof.

In some implementations the therapeutic agent is linezolid, azirthromycin, or combination thereof.

In some implementations the therapeutic agent is amethocaine, lignocaine, oxbuprocaine, proxymetacaine, or combination thereof.

In some implementations the therapeutic agent is amethocaine, lignocaine, oxbuprocaine, proxymetacaine, or combination thereof.

In some implementations the therapeutic agent is ketorolac tromethamine, brimonidine, azelastine hydrochloride, azithromycin, brolucizumab-dbll, bepotastine besylate, besifloxacin, dorzolamide hydrochloride, cysteamine hydrochloride, difluprednate, satralizumab-mwge, aflibercept, loteprednol etabonate, tasimelteon, adalimumab, chloroprocaine hydrochloride, avacincaptab pegol, ocriplasmin, tebentafusp-tebn, ranibizumab, bimatoprost, voretigene neparvocec, perfluorohexyloctane, ofloxacin, phenylephrine, oralair, cenegermin-bkbj, dexamethasone, pilocarpine hydrochloride, unoprostone isopropyl, cyclosporine, netarsudil, latanoprost, travoprost, varenicline, valganciclovir hydrochloride, trifluridine, verteporfin, latanoprostene bunod, lotilaner, incobotulinum toxin A, lifitegrast, cetirizine, tafluprost, ganciclovir, gatofloxacin, or a combination thereof.

The therapeutic agent-loaded corneal tissue may be transplanted into a subject in need thereof. In some implementations, the therapeutic agent-loaded corneal tissue may be transplanted in a full-thickness transplant, a partial endothelial transplant, or a partial epithelial transplant.

The therapeutic agent-loaded corneal tissue may be used in a variety of keratoplasties. In some implementations, the therapeutic agent-load corneal tissue is transplanted in a penetrating keratoplasty. In some implementations, the therapeutic agent-load corneal tissue is transplanted in a tectonic keratoplasty. In some implementations, the therapeutic agent-load corneal tissue is transplanted in a therapeutic keratoplasty, for example full thickness therapeutic keratoplasty, patch graft, or deep anterior lamellar keratoplasty. In some implementations, the therapeutic agent-load corneal tissue is transplanted to eliminate an infective load from the eye. In some implementations, the therapeutic agent-load corneal tissue is transplanted in a cosmetic keratoplasty. In some implementations, the therapeutic agent-load corneal tissue is transplanted in an optical keratoplasty.

Also disclosed herein are transplantable corneal tissues suitable for transplant. As used herein, corneal tissue suitable for transplant refers to tissues that may transplanted into a subject in need thereof. Such tissues may also be designated ex vivo corneal tissues. The attending physician may punch a suitable button in the ex vivo corneal tissue and deliver it to a patient in need thereof using the appropriate surgical technique. In some implementations the ex vivo corneal tissue includes all of the five aforementioned tissue layers. In some implementations the ex vivo corneal tissue includes only epithelium. In some implementations the ex vivo corneal tissue includes only epithelium and Bowman's layer. In some implementations the ex vivo corneal tissue includes only epithelium, Bowman's layer stroma, and stroma. In some implementations the ex vivo corneal tissue includes only epithelium, Bowman's layer stroma, stroma, and Descemet's membrane. In some implementations the ex vivo corneal tissue includes only endothelium. In some implementations the ex vivo corneal tissue includes only endothelium and Descemet's membrane. In some implementations the ex vivo corneal tissue includes only endothelium, Descemet's membrane, and stroma. In some implementations the ex vivo corneal tissue includes only endothelium, Descemet's membrane, stroma, and Bowman's layer. As used herein, a given tissue is said to include only the specified layers when at least 85%, 90%, 95%, or 98% (by weight) of the tissue present corresponds to the specified layers.

In some implementations, the ex vivo corneal tissue can include an antibacterial, antifungal, antiviral, cycloplegic, antiglaucoma therapy, corticosteroid, immunosuppressant, dry-eye therapy, anesthetic, anti-inflammatory, anti-allergy, β-adrenoceptor antagonist, miotic, carbonic anhydrase inhibitor, topoisomerase inhibitor, prostaglandin, aldose reductase inhibitor, growth factor, collagenase inhibitor, or combination thereof.

In some implementations, the ex vivo corneal tissue not include gentamicin or amphotericin B.

In some implementations, the ex vivo corneal tissue can include betamethasone, clobetasone, dexamethasone, fluorometholone, hydrocortisone, prednisolone, or combination thereof.

In some implementations, the ex vivo corneal tissue can include antazoline, bromfenac, diclofenac, indomethacin, lodoxamide, saprofen, sodium cromoglycate, or combination thereof.

In some implementations, the ex vivo corneal tissue can include amethocaine, lignocaine, oxbuprocaine, proxymetacaine, or combination thereof.

In some implementations, the ex vivo corneal tissue can include cyclosporin, tacrolimus, or sirolimus.

In some implementations, the ex vivo corneal tissue can include pilocarpine, carbachol, physostigmine, or combination thereof.

In some implementations, the ex vivo corneal tissue can include carteolol, cetamolol, betaxolol, levobunolol, metipranolol, timolol, or combination thereof.

In some implementations, the ex vivo corneal tissue can include adrenaline, dipivefrine, or combination thereof.

In some implementations, the ex vivo corneal tissue can include acetazolamide, dorzolamide, or combination thereof.

In some implementations, the ex vivo corneal tissue can include topotecan, irinotecan, camptothecin, lamellarin D, etoposide, teniposide, doxorubicin, mitoxantrone, or amsacrine.

In some implementations, the ex vivo corneal tissue can include chloramphenicol, chlortetracycline, ciprofloxacin, framycetin, fusidic acid, gentamicin, neomycin, norfloxacin, ofloxacin, polymyxin, propamidine, tetracycline, tobramycin, quinolines, or combination thereof.

In some implementations, the ex vivo corneal tissue can include acyclovir, cidofovir, brincidofovir, ganciclovir, valacyclovir, trifluridine, trifluorothymidine, idoxuridine, foscarnet, interferons, or combination thereof.

In some implementations, the ex vivo corneal tissue can include pegagtanib sodium, ranibizumab, aflibercept or bevacizumab.

In some implementations, the ex vivo corneal tissue can include lidocaine, amethocaine, lignocaine, oxbuprocaine, proxymetacaine, or combination thereof.

In some implementations, the ex vivo corneal tissue can include moxifloxacin, gatifloxacin, tobramycin, amikacin, or combination thereof.

In some implementations, the ex vivo corneal tissue can include homotropine or atropine, or combination thereof.

In some implementations, the ex vivo corneal tissue can include natamycin, voriconazole, itraconazole, ketoconazole, or combination thereof.

In some implementations, the ex vivo corneal tissue can include linezolid, azirthromycin, or combination thereof.

In some implementations, the ex vivo corneal tissue can include amethocaine, lignocaine, oxbuprocaine, proxymetacaine, or combination thereof.

In some implementations, the ex vivo corneal tissue can include amethocaine, lignocaine, oxbuprocaine, proxymetacaine, or combination thereof.

In some implementations, the ex vivo corneal tissue can include ketorolac tromethamine, brimonidine, azelastine hydrochloride, azithromycin, brolucizumab-dbll, bepotastine besylate, besifloxacin, dorzolamide hydrochloride, cysteamine hydrochloride, difluprednate, satralizumab-mwge, aflibercept, loteprednol etabonate, tasimelteon, adalimumab, chloroprocaine hydrochloride, avacincaptab pegol, ocriplasmin, tebentafusp-tebn, ranibizumab, bimatoprost, voretigene neparvocec, perfluorohexyloctane, ofloxacin, phenylephrine, oralair, cenegermin-bkbj, dexamethasone, pilocarpine hydrochloride, unoprostone isopropyl, cyclosporine, netarsudil, latanoprost, travoprost, varenicline, valganciclovir hydrochloride, trifluridine, verteporfin, latanoprostene bunod, lotilaner, incobotulinum toxin A, lifitegrast, cetirizine, tafluprost, ganciclovir, gatofloxacin, or a combination thereof.

Depending on the specific therapeutic agent and condition to be treated, the ex vivo corneal tissue can include the therapeutic agent in an amount from 1 μg to 1 g. In some implementations, the ex vivo corneal tissue can include the therapeutic agent in an amount from 1-1,000 μg, from 1-50 μg, from 25-100 μg, from 50-250 μg, from 100-500 μg, from 250-750 μg, from 500-1,000 μg, from 1-1,000 mg, from 1-50 mg, from 25-100 mg, from 50-250 mg, from 100-500 mg, from 250-750 mg, or from 500-1,000 mg.

The therapeutic agent-loaded corneal tissue may be used to treat patients with a variety of conditions. In some implementations, the patient is diagnosed with pseudophakic bullous keratopathy (pseudophakic corneal edema), aphakic bullous keratopathy (aphakic corneal edema). In some implementations the patient is diagnosed with one or more corneal stromal dystrophies, including granular dystrophy, lattice dystrophy, macular dystrophy, cCentral crystalline dystrophy of Schnyder, or central cloudy dystrophy of Francois.

In some implementations the patient is diagnosed with one or more corneal endothelial dystrophies, including Fuchs dystrophy, congenital hereditary endothelial dystrophy, posterior polymorphous dystrophy, Iridocorneal endothelial syndrome, or Chandler syndrome.

In some implementations the patient is diagnosed with corneal ectasia, for example anterior keratoconus, posterior keratoconus, or keratoglobus.

In some implementations the patient is diagnosed with one or more congenital opacities: Peter anomaly, sclerocornea aniridia, or congenital glaucoma

In some implementations the patient is diagnosed with viral keratitis, for example herpes simplex, adenovirus, or herpes zoster. In some implementations the patient is diagnosed with microbial keratitis, for example bacterial keratitis, fungal keratitis, chlamydial keratitis, infectious crystalline keratopathy, trachoma, parasitic keratitis, or acanthamoeba. In some implementations the patient is diagnosed with fungal keratitis, including a fungal keratitis with corneal perforation, a fungal keratitis with scleral involvement, a fungal keratitis with rapid corneal melt, or descemetocele.

In some implementations the patient is diagnosed with pythium keratitis or keratomalacia.

In some implementations the patient is in need of corneal transplant due to acid or alkali burns.

In some implementations the patient is experiencing corneal degeneration, for example spheroidal degeneration, Terrien marginal degeneration, or Band shaped keratopathy. In some implementations the patient has a traumatic corneal scar. In some implementations, the patient has non-infectious ulcerative keratitis (non-infectious). In some implementations the patient has keratoconjunctivitis sicca, Sjogren syndrome, exposure keratopathy, neurotrophic or neuroparalytic keratopathy, or Mooren ulcer.

In some implementations the therapeutic agent-loaded corneal tissue may be transplanted in a repeat graft, for example a repeat graft due to graft rejection, post-TPK, or primary graft failure.

The therapeutic agent-loaded corneal tissue may be transplanted using any known surgical technique. In some implementations, the corneal tissue is delivered via a full tissue transplantation. In some implementations, the corneal tissue is delivered via a partial thickness transplant. In some implementations, the corneal tissue is delivered via descemetorhexis without endothelial keratoplasty (DWEK), Descemet stripping only (DSO), deep lamellar endothelial keratoplasty (DLEK), Descemet stripping endothelial keratoplasty (DSEK), Descemet stripping automated endothelial keratoplasty (DSAEK), Descemet membrane endothelial keratoplasty (DMEK), pre-Descemet endothelial keratoplasty (PDEK), and Descemet membrane endothelial transfer (DMET).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method of preparing an ex vivo corneal tissue, comprising loading a therapeutic agent into a corneal tissue.

2. The method of claim 1, wherein the corneal tissue is an autologous corneal tissue, a donor corneal tissue, a reconstructed corneal tissue, or a combination thereof.

3. The method of claim 1, wherein the therapeutic agent is loaded into epithelium tissue, Bowman's layer stroma tissue, stroma tissue, Descemet's membrane tissue, endothelium tissue, or a combination thereof

4. The method of claim 1, wherein the therapeutic agent is loaded into the corneal tissue using microneedles, electroporation, surgical incision, solution equilibration, or a combination thereof.

5. The method of claim 4, comprising contacting the corneal tissue with a microneedle array comprising the therapeutic agent.

6. The method of claim 4, comprising disposing the corneal tissue in a solution comprising the therapeutic agent, and optionally applying an electromagnetic pulse to the corneal tissue.

7. The method of claim 4, comprising making an incision in the corneal tissue and applying the therapeutic agent to the exposed area.

8. The method of claim 1, wherein the therapeutic agent comprises an antibacterial, antifungal, antiviral, cycloplegic, antiglaucoma therapy, corticosteroid, immunosuppressant, dry-eye therapy, anesthetic, anti-inflammatory, anti-allergy, β-adrenoceptor antagonist, miotic, carbonic anhydrase inhibitor, topoisomerase inhibitor, prostaglandin, aldose reductase inhibitor, growth factor, collagenase inhibitor, or combination thereof.

9. The method of claim 1, wherein the therapeutic agent does not comprise gentamicin or amphotericin B.

10. The method of claim 1, further comprising transplanting the ex vivo corneal tissue into a subject in need thereof.

11. The method of claim 10, wherein the transplanting comprises a full thickness transplant, a partial epithelial transplant, or a partial endothelial transplant.

12. The method of claim 10, wherein the transplanting comprises descemetorhexis without endothelial keratoplasty, Descemet stripping only, deep lamellar endothelial keratoplasty, Descemet stripping endothelial keratoplasty, Descemet stripping automated endothelial keratoplasty, Descemet membrane endothelial keratoplasty, pre-Descemet endothelial keratoplasty, or Descemet membrane endothelial transfer.

13. An ex vivo corneal tissue, obtained by the method of claim 1.

14. An ex vivo corneal tissue, comprising at least one therapeutic agent.

15. The ex vivo corneal tissue of claim 14, comprising epithelium tissue, Bowman's layer stroma tissue, stroma tissue, Descemet's membrane tissue, endothelium tissue, or a combination thereof.

16. The ex vivo corneal tissue of claim 14, wherein the tissue comprises only endothelium tissue, only epithelium tissue, or only Bowman's layer stroma tissue, stroma tissue, and Descemet's membrane tissue.

17. The ex vivo corneal tissue of claim 14, wherein the therapeutic agent comprises an antibacterial, antifungal, antiviral, cycloplegic, antiglaucoma therapy, corticosteroid, immunosuppressant, dry-eye therapy, anesthetic, anti-inflammatory, anti-allergy, β-adrenoceptor antagonist, miotic, carbonic anhydrase inhibitor, topoisomerase inhibitor, prostaglandin, aldose reductase inhibitor, growth factor, collagenase inhibitor, or combination thereof.

18. The ex vivo corneal tissue of claim 14, wherein the therapeutic agent does not comprise gentamicin or amphotericin B.

19. A method of performing a keratoplasty in a subject in need thereof, comprising transplanting the ex vivo corneal tissue of claim 14 in the subject in need thereof.

20. The method of claim 19, wherein the subject is diagnosed with pseudophakic bullous keratopathy, aphakic bullous keratopathy, corneal stromal dystrophies, corneal endothelial dystrophies, corneal ectasia, congenital opacity, viral keratitis, microbial keratitis, fungal keratitis, pythium keratitis keratomalacia, acid burns, alkali burns, corneal degeneration, traumatic corneal scar, non-infectious ulcerative keratitis, keratoconjunctivitis sicca, Sjogren syndrome, exposure keratopathy, neurotrophic keratopathy, neuroparalytic keratopathy, or Mooren ulcer.
